(19) United States
(12) Patent Application Publication
(10) Pub. No.: US 2014/0377714 A1
(43) Pub. Date: Dec. 25, 2014

(54) SCAN BODY FOR DETERMINATION OF POSITIONING AND ORIENTATION OF A DENTAL IMPLANT

(71) Applicant: nt-trading GmbH & Co. KG, Karlsruhe (DE)

(72) Inventor: Dirk JAHN, Weyher (DE)

(73) Assignee: nt-trading GmbH & Co. KG, Karlsruhe (DE)

(21) Appl. No.: 14/011,936

(22) Filed: Aug. 28, 2013

(30) Foreign Application Priority Data
Jun. 20, 2013 (DE) 20 2013005821.8

(51) Int. Cl. A61C 1/08; A61C 8/00; A61K 6/083; A61B 6/14

(52) U.S. Cl. CPC A61C 1/084; A61B 6/145; A61C 8/0089; A61K 6/083
USPC 433/29

(57) ABSTRACT

A scan body for determination of a positioning and orientation of a dental implant, including a base part having an interface, including a three-dimensional scan region comprising at least four different scan areas, including a transition region axially between the scan region and the interface, as well as including a fastening screw for fixing the scan body in the implant, characterized in that the base part comprising the interface on the one hand and a scan element comprising the scan region including the transition region on the other hand are separately produced components, which are connected to one another by means of a non-positive and/or a positive connection.

### CROSS REFERENCE TO RELATED APPLICATION

The invention claims priority to German Utility Model DE 202013005821.8, filed Jun. 20, 2013, the disclosure of which is incorporated by reference herein in its entirety.

### FIELD OF INVENTION

The invention relates to a scan body for determination of a positioning and orientation of a dental implant, including a base part comprising an interface, including a three-dimensional scan region comprising at least four different scan areas, including a transition region axially between the scan region and the interface, as well as including a fastening screw for fixing the scan body in the implant.

### DESCRIPTION OF THE RELATED ART

Such a scan body is known from EP 2 457 536 A2. Said known scan body comprises a base part, a transition region as well as a scan region, wherein the scan region comprises four different scan areas distributed over the circumference of said region. The base part including a corresponding interface used for fitting into the corresponding dental implant is integrated in a one-piece component. Only the fastening screw is separated from said component and is inserted into a corresponding passage of the scan body from above, in order to allow fixing inside the dental implant.

The object of the invention is to provide a scan body of the above type which has an improved field of application as compared to the prior art.

### SUMMARY OF THE INVENTION

Said object is achieved in that the base part comprising the interface on the one band and a scan element comprising the scan region including the transition region on the other hand are separately produced components which are connected to one another by means of a non-positive and/or a positive connection. The separate production of the base part on the one hand and of the scan element having the scan region and the transition region on the other hand allows manufacturing of the different components from different materials. Furthermore, as a result of the separation of the base part on the one hand and of the scan element on the other hand, there is an option to combine the component forming the scan region and the transition region with different base parts, each having different interface designs. That allows to hold available a modular scan body with multiple base parts of correspondingly different design, and thus, depending on the implant system, to connect a base part with a accordingly adapted interface to the component forming the scan region and the transition region. That is particularly advantageous, since different implant systems have different interface designs to which the scan body has to be adapted in each case. Thus, it is not required to provide different configurations for the component forming the scan region and the transition region, in fact, equal parts can be used in each case.

In one embodiment of the invention, an external contour and an internal contour of a receptacle of the scan element have complementary axial guiding profiles with respect to a central longitudinal axis of the scan body, which profiles ensure an insertion in precise position and an anti-rotation securing of the base part relative to the receptacle. An exact and anti-rotation protected alignment of the interface relative to the component comprising the interface is required for a reliable and significant scan procedure. The guiding profiles complementary to one another ensure a connection of the base part to the other component, namely the scan element, which comprises the scan region and the transition region, in a clearly defined and constant alignment, resulting in insertion in precise position of the base part into the receptacle of the scan region and of the transition region.

As a result of the fact that the scan region of the scan body comprises four or more different scan areas, it is sufficient when only one individual scan area is detected during a scan procedure. The rest of the different scan areas can be defined subsequently in a simple manner via a reference body. That allows a position and an orientation of the dental implant within the corresponding jawbone of a patient and in relation to corresponding adjacent dental regions of the jawbone with high precision.

In a further embodiment of the invention, the internal contour of the receptacle of the scan element and/or the external contour of the base part comprise mutually complementary, positive-connected snap profiles axially operative in order to allow a non-loosening secured assembly of the base part in the scan element. Inserting the base part into the receptacle of the scan element causes a snap-locking between the two components, which prevent an inadvertent detaching of the base part from the component comprising the scan region and the transition region.

In a further embodiment of the invention, the fastening screw is held in the receptacle of the scan element in a captivated manner. The fastening screw is axially secured in the base part and is inserted into the component comprising the scan region and the transition region together with the base part. The scan element has an assembly access coaxially relative to the alignment of the fastening screw, through which access a tool can operate a screw head of the fastening screw, which head is provided with tool engaging surfaces. The cross-section of the assembly access is smaller than a cross-section of the screw head, so that in fact the fastening screw is held in an axially movable, but captivated manner within the receptacle of the scan region and of the transition region between the base part and assembly access. That provides a substantially simplified handling of the scan body during assembly or disassembly.

In a further embodiment of the invention, the base part is produced of a metal. Advantageously, the base part including the interface is produced from titanium. In contrast, the component comprising the scan region and the transition region is preferably produced from a thermoplastic synthetic material, in particular polyetheretherketone (PEEK).

### BRIEF DESCRIPTION OF THE DRAWINGS

Further advantages and features of the invention result from the claims as well as from the subsequent description of a preferred exemplary embodiment of the invention, which is illustrated by means of the drawings.

FIG. 1 shows a view from below of an embodiment of a scan body according to the invention,

FIG. 2 shows the scan body according to FIG. 1 in a side view, and

FIG. 3 shows a longitudinal sectional view along a section plane through the scan body according to FIG. 2, and

FIG. 4 shows a cross-section through the scan body along the section line IV-IV in FIG. 3.

### DETAILED DESCRIPTION THE INVENTION

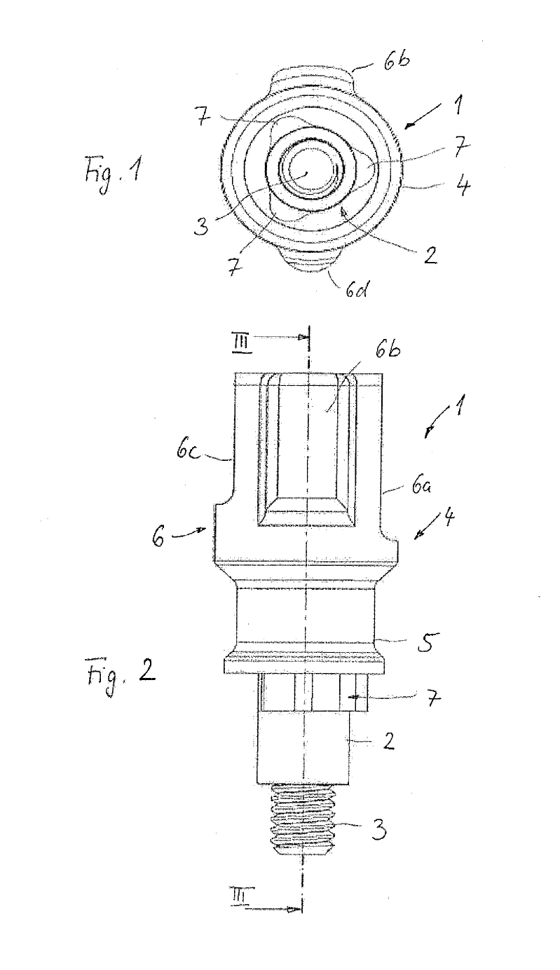

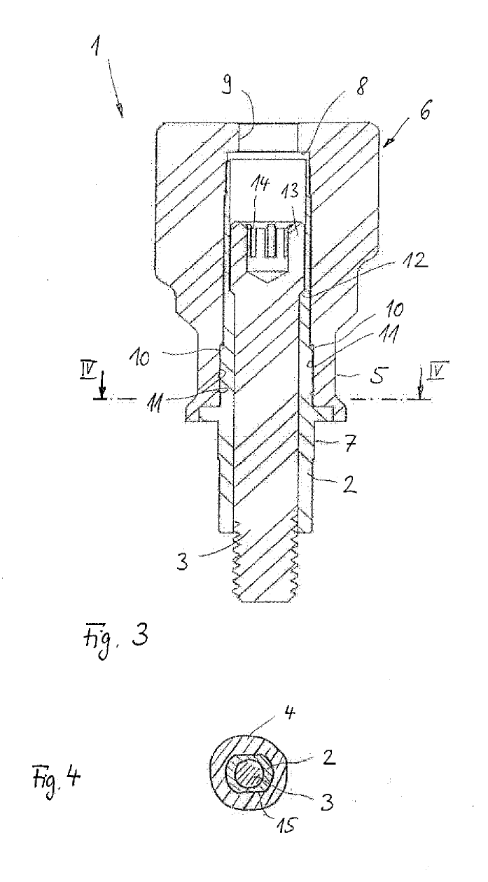

A scan body 1 according to FIGS. 1 to 3 is provided as to determine intraorally a positioning and orientation of a dental implant fixed in a jawbone. For that purpose, the scan body is inserted in a corresponding dental implant in the mouth region of a patient. After that, a corresponding scan procedure is effected by means of a scan system. The detected scan result is compared to data of a reference scan body of an electronic storage library. Said comparison allows a particularly exact determination of the alignment of the dental implant in particular also in relation to the gingiva, to adjacent teeth, to the jawbone region or to adjacent dental prostheses or dental implants. As a result, the designs of abutments and dental prostheses can particularly exactly and individually be adjusted in response to the results of corresponding scan procedures. The scan body 1 comprises four different scan areas 6a to 6d distributed over its scan region 6. The intraoral detection of already one individual scan area allows the determination of the exact alignment using the data of the reference scan body, as described above.

The scan body 1 is composed of three parts. A first body element is produced from a plastic material, here polyetheretherketone, as a scan element 4 and comprises the scan region 6 as well as a transition region 5 facing towards a base part 2. The scan region 6 and the transition region 5 are integrated in one piece in the scan element 4. The base part 2 is made of metal, preferably titanium or a titanium alloy, and is configured essentially in the form of a sleeve. The base part 2 is provided with an interface 7, which is molded integrally to the base part 2. The interface 7 comprises interface profiles projecting outwards from a shell of the base part 2, which profiles are matched to complementary interface profiles of the corresponding dental implant into which the scan body 1 is to be inserted. Dental implants have differently configured interface profiles depending in each case on the implant system the dental implants were chosen from. The base part 2 has interface profiles of an interface 7 which are matched to the respective dental implant system.

In the base part 2, a fastening screw 3 is accommodated, which is supported by means of a screw head 13 axially downwards, i.e. towards the interface 7, with a positive connection by means of a circumferential support collar 12 of an internal contour of the base part 2. The screw head 13 of the fastening screw 3 is provided with a blind hole that extends axially inwards from above, in which hole tool engaging surfaces 14 are integrated. The one-piece base part is open towards an upper end face in a sleeve-type manner such that the fastening screw 3 can be inserted into the base part 2 from above and that said screw can reach through the base part 2. A lower region of the fastening screw 3, which region is opposite to the screw head 13, is provided with an outer thread, which corresponds to an inner thread of the dental implant, in order to allow a screwing in of the fastening screw 3 into the dental implant. Above the interface 7, the base part 2 is circumferentially provided with a annular collar that projects outwards, which is supported on the end face side on a corresponding annular shoulder in the region of a lower end face side of the scan element 4. A lower, cylindrical sleeve section of the base part 2, to which the interface 7 is adjacent towards the support collar, is inserted into a corresponding receiving space of the dental implant in the assembled state, wherein the interface 7 ensures an anti-rotation securing of the base part 2 in the dental implant.

In order to be able to insert the base part 2 into the scan element 6, the scan element 6 is provided with a cylinder-type receptacle 8 open towards the bottom side. An internal contour of the receptacle 6 and an external contour of the base part 2 above the annular collar have mutually complementary, axial guide faces 15 (FIG. 4), in order to allow an axial insertion of the base part 2 exactly aligned into the rotation direction. Suitable axial guide faces 15 are planar, mutually complementary surface sections in the region of the external contour of the base part 2 as well as in the region of the internal contour of the receptacle 8. The receptacle 8 does not have an exactly cylindrical course of the internal contour, but rather an internal contour that slightly tapers conically upwards starting from the annular shoulder. Corresponding thereto, also the external contour of the base part 2 above the annular collar is not exactly cylindrical, but rather configured in a slightly conically tapering manner. Furthermore, the external contour of the base part 2 is provided with a snap profile projecting slightly radially outwards, to which profile a complementary snap profile 10 is assigned on the internal contour of the receptacle 8. The snap profiles 11 and 10 axially engage into one another upon axial insertion of the base part 2 up to the stop of its annular collar on the annular shoulder of the scan element 6. The fastening screw 3 is supported axially movable and rotatingly movable inside the base part 2.

In order to allow actuation of the fastening screw from above in the inserted state of the base part 2, the scan element 4 is provided with a through-hole 9 in the region of the scan element end face facing away from the annular shoulder, the cross section of which hole is smaller than a cross-section of the screw head 13. However, the cross-section of the through-hole 9 is sufficiently large to allow penetration of a tool to the tool engaging surfaces 14 of the screw head 13. As a result, the screw head 13 is held in the scan element 4 in an upwards captivated manner. The base part 2 is also held captivated in the scan element 4 due to the engaging in the region of the snap profiles 10, 11.

The scan element 4 comprises a scan region 6 which is provided with a total of four different scan areas 6a to 6d distributed over the circumference of said region. Then scan region 6 is configured radially thicker as compared to the transition region 5. The scan region 6 comprises two opposite and radially outwards projecting scan areas 6b, 6d, which are configured in the form of cams. In this case, the opposite scan areas 6b and 6d are differently contoured as well as differently configured in terms of their width and their axial length in relation to a central longitudinal axis of the scan body 1. Turned by 90° in the rotation direction relative to the scan areas 6b and 6d, two scan areas 6a and 6c are provided which are configured as planar scan areas 6a, 6c radially offset inwards in relation to a cylinder periphery of the scan region 6a. The axial length of the opposite scan areas 6a, 6c is different relative to one another. The scan areas also have a differing width.

For assembling of the scan body 1, the scan element 4 including the transition region 5 and the scan region 6 is provided as a one-piece component, which is composed of a plastic material, here PEEK. Furthermore, a base part 2 with a suitable interface 7 which part is matched to the respective dental implant, into which the scan body 1 is to be inserted, is selected, wherein the base part 2 including the interface 7 is produced as a one-piece component made of titanium or a titanium alloy. In addition, to the base part 2 is assigned a fastening screw 3 that is matched to the inner thread of the respective dental implant, which screw is inserted axially into the base part 2 from above. The base part 2 together with the inserted fastening screw 3 is inserted axially into the receptacle 8 of the scan element 4 from below, wherein the axial guide surfaces of the base part 2 on the one hand and of the receptacle 8 of the scan element 4 on the other hand have to be in alignment relative to one another. In the inserted end position of the base part 2, the snap profiles 11 of the base part 2 and the snap profiles 10 of the receptacle 8 of the scan element 4 engage into one another, so that the base part 2 is fixed in the scan element 4. Then, the scan body 1 can be screwed into the respective dental implant by means of a corresponding tool which penetrates the through-hole 9 and which contacts the fastening screw 3 in the region of the screw head 13.

1. A scan body for determination of a positioning and orientation of a dental implant, including a base part (2) comprising an interface (7), including a three-dimensional scan region (6) comprising at least four different scan areas (6a to 6d), including a transition region (5) axially between the scan region (6) and the interface (7), as well as including a fastening screw (3) for fixing the scan body (1) in the implant, characterized in that the base part (2) comprising the interface (7) on the one hand and a scan element (4) comprising the scan region (6) including the transition region (5) on the other hand are separately produced components, which are connected to one another by means of a non-positive and/or a positive connection.

2. The scan body according to claim 1, characterized in that an external contour of the base part (2) and an internal contour of a receptacle (8) of the scan element (4) comprise complementary—with respect to a central longitudinal axis of the scan body (1)—axial guiding profiles (15) which ensure an insertion in precise position and an anti-rotation securing of the base part (2) relative to the receptacle (8).

3. The scan body according to claim 2, characterized in that the internal contour of the receptacle (8) of the scan element (4) and/or the external contour of the base part (2) comprise mutually complementary, axially positive-connected operative snap profiles (10, 11), in order to allow a non-loosening secured assembly of the base part (2) in the scan element (4).

4. The scan body according to claim 2, characterized in that the fastening screw (3) is held in the receptacle (8) of the scan element (4) in a captivated manner.

5. The scan body according to claim 1, characterized in that the scan element is made of a synthetic material, in particular of polyetheretherketone (PEEK), and that the base part is made of metal, in particular titanium or a titanium alloy.
